(19) United States
(12) Patent Application Publication
(10) Pub. No.: US 2018/0207193 A1
(43) Pub. Date: Jul. 26, 2018

(54) COMPOSITIONS AND METHODS FOR IMPROVING ENZYME REPLACEMENT THERAPY FOR LYSOSOMAL STORAGE DISEASES

(71) Applicant: DUKE UNIVERSITY, Durham, NC (US)

(72) Inventors: Priya S. KISHNANI, Durham, NC (US); Baodong SUN, Durham, NC (US)

(21) Appl. No.: 15/879,052

(22) Filed: Jan. 24, 2018

Related U.S. Application Data
(60) Provisional application No. 62/449,762, filed on Jan. 24, 2017.

(51) Int. Cl. A61K 31/713; A61K 47/54; A61P 3/00; A61K 38/43

(52) U.S. Cl. CPC A61K 31/713; A61K 38/43; A61P 3/00; A61K 47/549

(57) ABSTRACT

The present disclosure provides compositions and methods for improving the efficacy of enzyme replacement therapy for lysosomal storage diseases.

### CROSS-REFERENCE TO RELATED APPLICATIONS

This application claims priority to U.S. Provisional Patent Application No. 62/449,762, filed Jan. 24, 2017, the disclosure of which is hereby incorporated by cross-reference in its entirety.

### BACKGROUND

Pompe disease (glycogen storage disease type II, GSD II) is a lysosomal storage disorder caused by a deficiency of lysosomal enzyme acid-α-glucosidase (GAA; acid maltase), and characterized by progressive structural disruption and cell dysfunction of cardiac and skeletal muscles due to progressive accumulation of lysosomal glycogen in these tissues (Kishnani et al. (2012) Am J Med Genet C Semin Med Genet 160C(1): 1-7). Cation-independent mannose-6-phosphate receptor (CI-M6PR)-mediated enzyme replacement therapy (ERT) with recombinant human GAA (rhGAA, Alglucosidase alfa, Myozyme) is an FDA approved therapy for Pompe disease and has been effective in improving cardiomyopathy and overall survival and daily activities for some patients with Pompe disease (Nicolino et al. (2009) Genet. Med. 11(3):210-219; Kishnani et al. (2007) Neurology 68(2):99-109).

However, the high abundance of M6PR on the surface of liver cells results in absorbance of the majority of administered enzyme by the liver, a non-affected tissue for Pompe disease and for most other lysosomal storage diseases (LSDs). The poor enzyme uptake by muscle tissues in Pompe disease requires frequent intravenous infusions of high doses of rhGAA to patients with Pompe disease to achieve therapeutic efficacy, which significantly increases treatment cost and the likelihood of developing severe immune responses including high titers of anti-drug antibody and infusion-associated reactions. At this time the dose for rhGAA is up to 40 mg/kg/week to prevent motor decline; this is four-fold higher than the recommended dose of 20 mg/kg/every 2 weeks.

RNA interference (RNAi) has broad potential as a therapeutic to reversibly silence any gene. As described herein, a liver-restricted RNAi-mediated gene silencing approach to suppress M6PR expression in liver and improve rhGAA delivery to muscle tissues in Pompe disease patients is tested in Pompe disease mice (GAA-KO, 6^{neo/neo}) (Raben et al. (1998) J. Biol. Chem. 273(3):19086-19092). In particular, M6PR-specific short interfering RNA (siRNA) candidates are synthesized and chemically conjugated to a sugar molecule GalNAc (GalNAc-siRNA conjugates) for liver-targeting. As described herein, liver-specific down-regulation of M6PR expression increases the efficiency of rhGAA delivery to muscle tissues and improves the efficacy of ERT for Pompe disease, as well as M6PR-mediated ERT for other lysosomal storage diseases.

### BRIEF SUMMARY OF THE INVENTION

The present disclosure relates to methods and compositions of improving the efficacy of enzyme replacement therapy “ERT” in a subject.

One aspect of the present invention provides a method of improving the efficacy of an enzyme replacement therapy for a lysosomal storage disease in a subject comprising administering to the subject a therapeutically effective amount of a therapeutic candidate such that the efficacy of the enzyme replacement therapy is enhanced.

In some embodiments, the therapeutic candidate is RNAi polynucleotides or small molecule drugs. In other embodiments, the therapeutic candidate is selected from the group consisting of double stranded RNA, antisense oligonucleotides (ASO), small interfering RNA (siRNA), short hairpin RNA (shRNA), microRNA oligonucleotides (miRNAs), and small molecule drugs.

In some embodiments, the therapeutic candidate can be delivered to liver cells with a viral vector, wherein the viral vector is an adenoviral vector, adeno-associated viral (AAV) vector, retrovirus vector, hemagglutinating virus of Japan (HVJ) vector, lentiviral vector, and hepatitis B virus vector.

In some embodiments, the therapeutic candidate is conjugated to one or more moieties used to target the therapeutic candidate to liver cells or is encapsulated in a delivery vehicle. In other embodiments, the delivery vehicle is conjugated to one or more moieties used to target the therapeutic candidate to liver cells. In yet other embodiments, the delivery vehicle is a PEGylated liposome or nanoparticle, oligonucleotide nanoparticle, cyclodextrin polymer (CDP)-based nanoparticle, biodegradable polymeric nanoparticle formulated with poly-(D,L-lactide-co-glycolide) (PLGA), Poly-lactic acid (PLA), or N-(2-hydroxypropyl)methacrylamide (HPMA), lipid nanoparticle (LNP), stable nucleic acid lipid particle (SNALP), or vitamin A coupled lipid nanoparticle.

In some embodiments, the therapeutic candidate is chemically conjugated to one or more moieties that bind to an asialoglycoprotein receptor (ASGPR), an apolipoprotein, or a glycosaminoglycan. In other embodiments, the therapeutic candidate is chemically conjugated to one or more N-acetylgalactosamine (GalNAc) molecules.

In some embodiments, the therapeutic candidate is capable of down-regulating the expression of mannose-6-phosphate receptor (M6PR) in a liver-specific manner in the subject. In other embodiments, the therapeutic candidate is an M6PR-specific short interfering RNA (siRNA) chemically conjugated to GalNAc.

In some embodiments, the lysosomal storage disease is Fabry disease, Gaucher disease, MPS diseases including MPS I (Hurler, Hurler-Scheie, or Scheie syndrome), MPS II (Hunter disease), and MPS VI (Maroteaux-Lamy syndrome), Pompe disease, Niemann Pick B, Batten, or Wolman disease.

In some embodiments, the therapeutic candidate is administered intravenously, subcutaneously, transdermally, intradermally, intramuscularly or orally. In other embodiments, the therapeutic candidate is administered concurrently with, prior to, or subsequent to the enzyme replacement therapy in the subject. In yet other embodiments, the therapeutic candidate is administered concurrently with, prior to, or subsequent to the enzyme replacement therapy with rhGAA in the subject. In yet other embodiments, the therapeutic candidate and the enzyme replacement therapy are administered to the subject on an effective dose level and dosing interval basis.

In other embodiments, the therapeutic candidate is administered concurrently with, prior to, or subsequent to the enzyme replacement therapy with rhGAA in the subject, wherein the therapeutic candidate is an M6PR-specific siRNA chemically conjugated to GalNAc or an M6PR-specific siRNA encapsulated in a delivery vehicle.

Another aspect of the present invention provides a method of inhibiting expression of M6PR mRNA comprising administering to a subject undergoing an enzyme replacement therapy for a lysosomal storage disorder an effective amount of a therapeutic candidate, wherein the therapeutic candidate comprises an M6PR-specific RNAi polynucleotide.

In some embodiments, the therapeutic candidate is chemically conjugated to one or more N-acetylgalactosamine (GalNAc) molecules or encapsulated in a delivery vehicle.

### BRIEF DESCRIPTION OF THE DRAWINGS

The foregoing aspects and other features of the invention are explained in the following description, taken in connection with the accompanying drawings, wherein:

FIG. 1 is a schematic showing conjugation of siRNA to an asialoglycoprotein receptor ligand derived from N-acetylgalactosamine (GalNAc). Nair et al. (2014) J. Am. Chem. Soc., 136(49):16958-16961.

### DETAILED DESCRIPTION OF THE INVENTION

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Articles “a” and “an” are used herein to refer to one or to more than one (i.e. at least one) of the grammatical object of the article. By way of example, “an element” means at least one element and can include more than one element.

As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which this disclosure belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of aspects and embodiments are disclosed. Each of these has individual benefit and each can also be used in conjunction with one or more, or in some cases all, of the other disclosed aspects and embodiments, whether specifically delineated or not. Accordingly, for the sake of clarity, this description will refrain from repeating every possible combination of the individual aspects and embodiments in an unnecessary fashion. Nevertheless, the specification and claims should be read with the understanding that such combinations are implicitly disclosed, and are entirely within the scope of the invention and the claims, unless otherwise specified.

One aspect of the present disclosure provides a method of improving the efficacy of an enzyme replacement therapy for a lysosomal storage disease in a subject comprising, consisting of, or consisting essentially of administering to the subject a therapeutically effective amount of a therapeutic candidate such that the efficacy of the enzyme replacement therapy is enhanced.

The terms, “improve,” “enhance,” “prevent,” or “reduce,” as used herein, indicate values that are relative to a baseline measurement, such as a measurement in the same individual prior to initiation of the treatment described herein, or a measurement in a control individual (or multiple control individuals) in the absence of the treatment described herein.

The therapeutic candidate is any therapeutic that is able to down-regulate the expression of mannose-6-phosphate receptor (M6PR) in a liver-specific manner in the subject. Examples include, but are not limited to, RNA interference (RNAi) polynucleotides and small molecule drugs.

The term “RNA interference (RNAi) polynucleotide” as used herein refers to a molecule capable of inhibiting or reducing gene expression or translation, by neutralizing targeted mRNA. Examples of an RNA interference (RNAi) polynucleotide include, but are not limited to, double stranded RNA (dsRNA), antisense oligonucleotides (ASO), small interfering RNA (siRNA), short hairpin RNA (shRNA), microRNA (miRNAs) oligonucleotides, and aptamers, and the like.

The terms “polynucleotide” and “oligonucleotide” are used interchangeably herein to refer to sequences with conventional nucleotide bases, sugar residues and internucleotide phosphate linkages, but also those which contain modifications of any or all of these moieties. The term “nucleotide” as used herein include those moieties which contain not only the natively found purine and pyrimidine bases adenine (A), guanine (G), thymine (T), cytosine (C), and uracil (U), but also modified or analogous forms thereof. Polynucleotides include RNA and DNA sequences of more than one nucleotide in a single chain. Modified RNA or modified DNA, as used herein, refers to a molecule in which one or more of the components of the nucleic acid, namely sugars, bases, and phosphate moieties, are different from that which occurs in nature.

Design, synthesis, and purification of RNA interference (RNAi) polynucleotides can be performed by established methods known in the art.

The term “double-stranded RNA (dsRNA)” is RNA with two complementary strands, similar to the DNA found in all cells. dsRNA forms the genetic material of some viruses (double-stranded RNA viruses). Double-stranded RNA such as viral RNA or siRNA can trigger RNA interference in eukaryotes, as well as interferon response in vertebrates.

The term “antisense oligonucleotides (ASO)” as used herein refer to the use of a nucleotide sequence, complementary by virtue of Watson-Crick base pair hybridization, to a specific mRNA to inhibit its expression and then induce a blockade in the transfer of genetic information from DNA to protein. The ASO molecule can be complementary to a portion of the coding or noncoding region of an RNA molecule, e.g., a pre-mRNA or mRNA. An ASO molecule of the invention can be, for example, about 10 to 25 nucleotides in length. An ASO molecule can be constructed using chemical synthesis and/or enzymatic ligation reactions using procedures known in the art. Alternatively, the ASO molecule can be transcribed biologically using an expression vector into which a nucleic acid has been subcloned in an antisense orientation (i.e., RNA transcribed from the inserted nucleic acid will be of an antisense orientation to a target nucleic acid of interest).

The term “small interfering RNA (siRNA),” also known as short interfering RNA or silencing RNA, is used herein to refer to a class of double-stranded RNA molecules, approximately 10-50 base pairs in length, but preferably 19-25 base pairs in length that interferes with the expression of specific target genes with complementary nucleotide sequences by degrading mRNA after transcription, preventing translation. An siRNA can have a nucleotide sequence identical (perfectly complementary) or substantially identical (partially complementary) to a portion of the coding sequence in an expressed target gene or RNA within the cell. An siRNA may have short 3′ overhangs. An siRNA may be composed of two annealed polynucleotides or a single polynucleotide that forms a hairpin structure. An siRNA molecule of the invention comprises a sense region and an antisense region. In one embodiment, the siRNA of the invention is assembled from two oligonucleotide fragments wherein one fragment comprises the nucleotide sequence of the antisense strand of the siRNA molecule and a second fragment comprises the nucleotide sequence of the sense region of the siRNA molecule. In certain embodiments, the siRNA are chemically modified. In another embodiment, the sense strand is connected to the antisense strand via a linker molecule, such as a polynucleotide linker or a non-nucleotide linker. An siRNA can be constructed using chemical synthesis and/or enzymatic ligation reactions using procedures known in the art. Alternatively, the siRNA nucleic acid can be transcribed biologically using an expression vector into which a nucleic acid has been subcloned in an antisense orientation (i.e., RNA transcribed from the inserted nucleic acid will be of an antisense orientation to a target nucleic acid of interest).

The term “short hairpin RNA (shRNA)” as used herein refers to an artificial RNA molecule with a tight hairpin turn that can be used to silence target gene expression via RNAi. shRNA is an advantageous mediator of RNAi because it has a relatively low rate of degradation and turnover. An shRNA can be constructed using chemical synthesis and/or enzymatic ligation reactions using procedures known in the art. Alternatively, the shRNA nucleic acid molecule can be transcribed biologically using an expression vector (plasmids or viral or bacterial vectors) into which a nucleic acid has been subcloned in an antisense orientation (i.e., RNA transcribed from the inserted nucleic acid will be of an antisense orientation to a target nucleic acid of interest).

The term “microRNA” as used herein refers to a small, non-coding RNA molecule (containing about 22 nucleotides) found in plants, animals and some viruses that functions in RNA silencing and post-transcriptional regulation of gene expression. Gene silencing may occur either via mRNA degradation or preventing mRNA from being translated. miRNAs resemble the siRNAs, except miRNAs derive from regions of RNA transcripts that fold back on themselves to form short hairpins, whereas siRNAs derive from longer regions of double-stranded RNA. An miRNA oligonucleotide can be constructed using chemical synthesis and/or enzymatic ligation reactions using procedures known in the art. Alternatively, the miRNA oligonucleotide can be transcribed biologically using an expression vector into which a nucleic acid has been subcloned in an antisense orientation (i.e., RNA transcribed from the inserted nucleic acid will be of an antisense orientation to a target nucleic acid of interest).

The term “aptamer” as used herein refers to short single-stranded oligonucleotides or a plurality of said oligonucleotides that bind to target molecules with high affinity, such as a small molecule, protein, nucleic acid, cell, tissue, or organism. Selection of aptamers that specifically bind a target mRNA may be accomplished by any suitable method known in the art, including but not limited to by an in vitro process known as whole Cell-SELEX (Systematic Evolution of Ligands by Exponential enrichment).

In other embodiments, the therapeutic candidate can target a specific tissue or cell, such as the liver, heart, muscle tissue, or tumor cells. Targeting moieties (targeting ligands) enhance the properties of a conjugate to which they are attached (e.g. the therapeutic candidate or a delivery vehicle comprising the therapeutic candidate) to improve cell-specific distribution and cell-specific uptake of the conjugate.

In some embodiments, the therapeutic candidate can target the liver through moieties that have high binding affinity for cell surface receptors found on the liver. In some embodiments, the therapeutic candidate is coupled to or chemically conjugated to one or more moieties (targeting ligands) that include peptides, antibodies, small molecules, glycans, phosphorothioate, and lectins used to target the therapeutic candidate to a cell surface receptor that is highly or abundantly expressed on hepatocytes.

Receptors that are abundantly expressed on the surface of hepatocytes include, but are not limited to, the asialoglycoprotein receptor (ASGPR), apolipoproteins, and glycosaminoglycans (GAGs). Examples of ASGPR-targeting moieties include, but are not limited to, lactose, galactose, N-acetylgalactosamine (GalNAc), galactosamine, N-formylgalactosamine, N-acetyl-galactosamine, N-propionylgalactosamine, N-n-butanoylgalactosamine, and N-iso-butanoyl-galactosamine. ASGPR-targeting moieties can be monomeric (e.g., having a single GalNAc) or multimeric (e.g., having multiple GalNAcs).

The terms “glycosaminoglycans,” “GAGs,” or “mucopolysaccharides” are used herein to refer to long, unbranched polysaccharides that contain a repeating disaccharide unit. Glycosaminoglycans include chondroitin sulfate (CS), dermatan sulfate, and heparan sulfate. Examples of GAG-targeting moieties include, but are not limited to, PTD-DRBD, a double-stranded RNA binding domain (DRBD) fused to the TAT protein transduction domain (PTD).

The term “apolipoproteins” as used herein refer to proteins that bind and transport lipids through the lymphatic and circulatory systems. There are six classes of apolipoproteins (apo), Apolipoprotein A, Apolipoprotein B, Apolipoprotein C, Apolipoprotein D, Apolipoprotein E, and Apolipoprotein H. There are also several apolipoprotein subtypes. Examples of apolipoproteins include, but are not limited to Apolipoprotein A1, Apolipoprotein A2, Apolipoprotein A4, Apolipoprotein A5, Apolipoprotein B48, Apolipoprotein B100, Apolipoprotein C1, Apolipoprotein C2, Apolipoprotein C3, and Apolipoprotein C4, Apolipoprotein D, Apolipoprotein E, and Apolipoprotein H.

In some embodiments, the therapeutic candidate is coupled or chemically conjugated to one or more moieties that target the liver. In some embodiments, the therapeutic candidate is chemically conjugated to one or more moieties that bind to the asialoglycoprotein receptor (ASGPR), apolipoproteins, or a glycosaminoglycan on liver cells. In some embodiments, the therapeutic candidate is chemically conjugated to one or more N-acetylgalactosamine (GalNAc) molecules. In other embodiments, the therapeutic candidate is an M6PR-specific RNAi polynucleotide that is chemically conjugated to one or more ASGPR-targeting moieties or one or more GAG-targeting moieties. In some embodiments, the therapeutic candidate is a M6PR-specific RNAi polynucleotide that is chemically conjugated to one or more GalNAc molecules. In other embodiments, the therapeutic candidate is a M6PR-specific short interfering RNA (siRNA) that is chemically conjugated to one or more GalNAc molecules.

In some embodiments, the liver-targeting moiety is directly coupled or chemically conjugated to an RNAi molecule or small molecule drug via a linker.

In other embodiments, the therapeutic candidate is encapsulated (packaged) in or attached to a delivery vehicle that is directly incorporated or chemically conjugated to one or more moieties that target the liver. The terms “delivery vehicle” or “delivery reagent” are used herein to refer to agents that allow the therapeutic candidate to navigate through the subject to reach the site of action without becoming degraded, inactivated, or comprised. Examples of delivery vehicles include, but are not limited to, Mirus Transit TKO lipophilic reagent, lipofectin, lipofectamine, cellfectin, polycations (e.g., polylysine), micelles, PEGylated liposome or nanoparticle, oligonucleotide nanoparticles, cyclodextrin polymer (CDP)-based nanoparticle, biodegradable polymeric nanoparticle formulated with poly-(D,L-lactide-co-glycolide) (PLGA), Poly-lactic acid (PLA), or N-(2-hydroxypropyl)methacrylamide (HPMA), lipid nanoparticle (LNP), stable nucleic acid lipid particle (SNALP), vitamin A coupled lipid nanoparticle, and combinations thereof.

In other embodiments, the therapeutic candidate is encapsulated in or attached to a delivery vehicle that is incorporated or chemically conjugated to one or more moieties that target the liver. In other embodiments, the therapeutic candidate is encapsulated in or attached to a delivery vehicle that is incorporated or chemically conjugated to one or more ASGPR-targeting moieties (e.g., GalNAc) or one or more GAG-targeting moieties. In other embodiments, a M6PR-specific RNAi polynucleotide is encapsulated in or attached to a delivery vehicle that is incorporated or chemically conjugated to one or more moieties that target the liver. In yet other embodiments, a M6PR-specific short interfering RNA (siRNA) is encapsulated in a delivery vehicle that is conjugated to one or more GalNac molecules. In yet other embodiments, a M6PR-specific short interfering RNA (siRNA) is encapsulated in a lipid nanoparticle that is conjugated to one or more moieties that target the liver.

The therapeutic candidate can also target a specific tissue or cell by passive targeting. For example, lipid particles (e.g, LNPs and SNALPs) target the liver without conjugation to a targeting moiety due to their size and surface composition. In some embodiments, the therapeutic candidate is encapsulated in a lipid particle. In other embodiments, a M6PR-specific RNAi polynucleotide is packaged in a LNP or SNALP. In yet other embodiments, a M6PR-specific short interfering RNA (siRNA) is encapsulated in a lipid nanoparticle.

In some embodiments, the therapeutic candidate is either complexed to positively charged polymers, dynamic polyconjugates, or lipids, or encapsulated in a delivery vehicle. In other embodiments, the delivery vehicle is directly incorporated or chemically conjugated to one or more moieties used to target the therapeutic candidate to a liver cell.

In other embodiments, the therapeutic candidate can be delivered to liver cells with a viral vector, including but not limited to, adenoviral vector, adeno-associated viral (AAV) vector, retrovirus vector, hemagglutinating virus of Japan (HVJ) vector, lentiviral vector, and hepatitis B virus vector.

The terms “mannose-6-phosphate receptor (M6PR),” “cation-independent mannose-6-phosphate receptor (CI-M6PR),” and “Insulin-like growth factor 2 receptor (IGF2R)” are used interchangeably herein to refer to the transmembrane glycoproteins that target therapeutic enzymes to lysosomes in vertebrates.

As used herein, the term “target mRNA” means human M6PR mRNA, mutant or alternative splice forms of human M6PR mRNA, or mRNA from cognate M6PR genes. As used herein, a gene or mRNA which is “cognate” to human M6PR is a gene or mRNA from another mammalian species, such as the rat and mouse, which is homologous to M6PR.

As used herein, therapeutic candidates comprising siRNA which is “targeted to the M6PR mRNA” means siRNA in which a first strand of the duplex has the same nucleotide sequence as a portion of the M6PR mRNA sequence. It is understood that the second strand of the siRNA duplex is complementary to both the first strand of the siRNA duplex and to the same portion of the M6PR mRNA. The siRNA of the invention can be targeted to any stretch of about 20 contiguous nucleotides (e.g, 15-29, 16-28, 17-27, 18-26, 19-25, 20-24, or 21-23 contiguous nucleotides) in any of the target mRNA sequences (the “target sequence”). Techniques for selecting target sequences for siRNA are known in the art. Thus, the sense strand of the siRNA comprises a nucleotide sequence identical to any contiguous stretch of about 20 contiguous nucleotides in the target mRNA. Generally, a target sequence on the target mRNA can be selected from a given cDNA sequence corresponding to the target mRNA, preferably beginning 50 to 100 nucleotides downstream (i.e., in the 3′ direction) from the start codon. The target sequence can, however, be located in the 5′ or 3′ untranslated regions, or in the region near the start codon.

As used herein, the term M6PR-specific RNAi polynucleotide refers to any RNAi polynucleotide (such as dsRNA, ASO, siRNA, shRNA, miRNAs, and aptamers) that is capable of down-regulating or inhibiting M6PR gene expression or translation.

The terms “down-regulate,” “inhibit,” or “knockdown” are used herein to refer to reducing the level of RNA transcribed from the target gene or the level of polypeptide, protein or protein subunit translated from the RNA, below the level which is observed in the absence of the blocking therapeutic candidate of the invention or below that level observed in the presence of a control inactive therapeutic candidate (e.g., containing a nucleic acid with a scrambled sequence or with inactivating mismatches). RNAi-mediated degradation of the target mRNA can be detected by measuring levels of the target mRNA or protein in the cells of a subject, using standard techniques for isolating and quantifying mRNA or protein.

The term “lysosomal storage disease” or “LSD” refers to the group of rare, inherited metabolic disorders that result from defects in lysosomal functions. Specifically, LSDs are caused by lysosomal dysfunction usually as a consequence of deficiency of a single enzyme required for the metabolism of lipids, glycoproteins (sugar-containing proteins), or mucopolysaccharides. They can also be caused due to defects in the transporter. Examples of LSDs include, but are not limited to, Fabry disease, Gaucher disease, mucopolysaccharidoses (MPS) diseases including MPS I (Hurler, Hurler-Scheie, or Scheie syndrome), MPS II (Hunter disease), and MPS VI (Maroteaux-Lamy syndrome), Pompe disease, Niemann Pick B, Batten, Wolman disease, and the like.

The terms “treating” or “treatment” as used herein refers to both therapeutic treatment and prophylactic or preventative measures. It refers to preventing, curing, reversing, attenuating, alleviating, minimizing, suppressing, or halting the deleterious effects of a disease state, disease progression, disease causative agent (e.g., bacteria or viruses), or other abnormal condition.

The terms “effective amount” or “therapeutically effective amount” as used herein refers to an amount of a therapeutic candidate sufficient to effect beneficial or desirable biological and/or clinical results. Such response may be a beneficial result, including, without limitation, amelioration, reduction, prevention, or elimination of symptoms of a disease or disorder. Therefore, the total amount of each active component of the therapeutic candidate is sufficient to demonstrate a meaningful benefit in the patient, including, but not limited to, improving the efficiency of recombinant protein delivery during ERT for treatment of lysosomal storage diseases and improving the efficiency of rhGAA delivery to muscle tissues and improve the efficacy of ERT for Pompe disease. A “therapeutically effective amount” may be administered through one or more preventative or therapeutic administrations. When the term “therapeutically effective amount” is used in reference to a single agent, administered alone, the term refers to that agent alone, or a composition comprising that agent and one or more pharmaceutically acceptable carriers, excipients, adjuvants, or diluents. When applied to a combination, the term refers to combined amounts of the active agents that produce the therapeutic effect, or composition(s) comprising the agents, whether administered in combination, consecutively, or simultaneously. The exact amount required will vary from subject to subject, depending, for example, on the species, age, and general condition of the subject; the severity of the condition being treated; and the mode of administration, among other factors known and understood by one of ordinary skill in the art. An appropriate “effective” amount in any individual case may be determined by one of ordinary skill in the art. Thus, a “therapeutically effective amount” will typically fall in a relatively broad range that can be determined through routine trials.

The therapeutic candidates described herein can be administered by any suitable route of administration. In certain embodiments, the therapeutic candidate is administered intravenously, subcutaneously, transdermally, intradermally, intramuscularly, orally, transcutaneously, intraperitoneally (IP), or intravaginally.

The therapeutic candidates of the invention can be administered to the subject either naked or in conjunction with a delivery reagent. Examples of delivery reagents for administration in conjunction with the therapeutic candidates include, but are not limited to, Minis Transit TKO lipophilic reagent, lipofectin, lipofectamine, cellfectin, polycations (e.g., polylysine), micelles, PEGylated liposome or nanoparticles, oligonucleotide nanoparticles, cyclodextrin polymer (CDP)-based nanoparticles, biodegradable polymeric nanoparticles formulated with poly-(D,L-lactide-co-glycolide) (PLGA), Poly-lactic acid (PLA), or N-(2-hydroxypropyl)methacrylamide (HPMA), lipid nanoparticles (LNP), stable nucleic acid lipid particles (SNALP), vitamin A coupled lipid nanoparticles, and combinations thereof.

One skilled in the art can also readily determine an appropriate dosage regimen for administering the therapeutic candidates to a given subject.

In another embodiment, the therapeutic candidate is formulated as pharmaceutical composition prior to administering to a subject, according to techniques known in the art. Pharmaceutical compositions of the present invention are characterized as being at least sterile and pyrogen-free. As used herein, “pharmaceutical formulations” or “pharmaceutical compositions” include formulations for human and veterinary use.

As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like. Preferably, the subject is a human patient is suffering from, or at risk of developing, a lysosomal storage disease.

As used herein, the term “enzyme replacement therapy (ERT)” refers to medical treatment which replaces an enzyme that is deficient or absent in the body. Lysosomal storage diseases are a primary application of ERT. There are around 50 lysosomal storage diseases with even more still being discovered. Lysosomal storage disorders arise because of genetic mutations that prevent the production of certain enzymes used in the lysosomes, which break down different macromolecules and proteins. The missing enzyme often leads to a build-up of the substrate within the body, resulting in a variety of symptoms, many of which are severe and can affect the skeleton, brain, skin, heart, and the central nervous system. Increasing the concentration of the missing enzyme within the subject has been shown to improve the subject's normal cellular metabolic processes and reduce substrate concentration in the subject. Currently, ERT is available for lysosomal storage diseases such as Gaucher disease, Fabry disease, MPS I, MPS II (Hunter syndrome), MPS VI, Niemann Pick type B, Batten disease, Pompe disease, and Wolman disease. ERT does not remedy the underlying genetic defect, but it increases the concentration of the enzyme that the subject is lacking.

The following examples are offered by way of illustration and not by way of limitation.

### EXAMPLE 1

### Design and Synthesis of GalNAc-siRNAs Targeting M6PR (IGF2R)

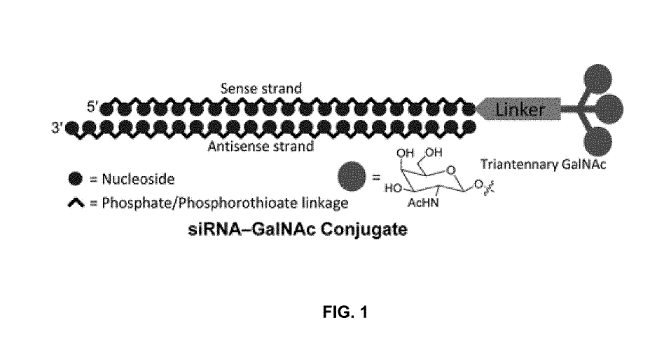

A potent and durable rodent cross-reactive GalNAc-siRNA targeting the cation-independent M6PR (IGF2R) is designed and synthesized and is screened for efficacy in appropriate in vitro systems (a representative schematic of a GalNAc-siRNA conjugate is shown in FIG. 1). From the in vitro screening set, approximately 4-6 siRNAs are evaluated in vivo in a single dose screen in WT mice for knockdown of M6PR. Based on the knockdown results of the single dose screen, 1-2 siRNAs are further characterized in vivo in a dose response and duration study in mice in which both mRNA and protein levels are assessed. C57 BL/6 mice are used for the siRNA selection and characterization studies. The dose level and dosing interval for studies in GAA-KO mice are guided by the results determined in this Example. Chronic dosing regimens of GalNAc-siRNAs to achieve sustained 80%, 50%, and 30% M6PR knockdown are then established.

### EXAMPLE 2

### Minimal Effective Dose of rhGAA in the Presence of Maximal GalNAc-siRNA-Mediated Knockdown of M6PR (IGF2R) in Liver

To determine the minimal effective dose of rhGAA in the presence of maximal GalNAc-siRNA-mediated knockdown of M6PR (IGF2R) in liver to achieve significant improvement of rhGAA uptake by skeletal muscles, a dose-ranging study is conducted that will be used for a long-term efficacy study. Three-month-old GAA-KO mice are first treated with GalNAc-siRNA at the maximal effective dose (e.g., ED80 as determined in Example 1) for liver-specific M6PR knockdown. After 7-10 days, rhGAA at varying doses is intravenously injected into these mice. This experiment includes 7 groups with n=5 mice per group (total 35 mice):

Group 1: rhGAA only at 20 mg/kg, once (standard ERT only controls)

Group 2: GalNAc-siRNA at ED80+rhGAA at 20 mg/kg, once

Group 3: GalNAc-siRNA at ED80+rhGAA at 10 mg/kg, once

Group 4: GalNAc-siRNA at ED80+rhGAA at 5 mg/kg, once

Group 5: GalNAc-siRNA at ED80+rhGAA at 2.5 mg/kg, once

Group 6: GalNAc-siRNA at ED80+rhGAA at 1 mg/kg, once

Group 7: No treatment controls

All mice are euthanized after 24 h following overnight fasting to collect tissues including liver, heart, quadriceps, gastrocnemius, and diaphragm. M6PR knockdown in liver, heart, and skeletal muscle is assayed by Q-RT-PCR and by Western blotting using anti-CI-M6PR mAb (ab124767, Abcam). GAA activity and glycogen content are analyzed in liver and all muscle tissues (Koeberl et al. (2011) Mol. Genet. Metab. 103(2):107-112; Sun et al. (2013) Mol. Genet. Metab. 108(2):145-147).

McVie-Wylie et al. reported that a 4-week treatment of GAA-KO mice with rhGAA at a weekly dose of 20 mg/kg significantly reduced glycogen in heart (−55% approximately) but had no effect on glycogen content in the skeletal muscle (quadriceps); in comparison, a higher dose (100 mg/kg) of rhGAA treatment significantly reduced glycogen levels in skeletal muscle. (2008) Mol. Genet. Metab. 94(4):448-45. It has also been demonstrated that a 12-week ERT with weekly rhGAA at 20 mg/kg in GAA-KO mice reduced glycogen levels in heart by 98% and in skeletal muscles by 33%-46% (paper in press). This suggests that a longer treatment of ERT (12-week) has a better efficacy in clearing glycogen accumulation in heart and skeletal muscle than the short-term (4-week) treatment. Therefore, in this experiment, an ERT only group (20 mg/kg, Group 1) is included as a reference of effective treatment of long-term ERT for comparison to each of the GalNAc-siRNA+rhGAA treatment groups (Groups 2-6). The minimal effective dose of rhGAA in the presence of maximal M6PR (IGF2R) knockdown that gives the equivalent effect to the ERT only group (20 mg/kg rhGAA, Group 1) is determined.

GAA activity values in muscles (heart and skeletal muscles) are used as the primary parameter to predict the effectiveness of long-term treatment. Glycogen content in heart (maybe skeletal as well) may be used as the second parameter. The results of this experiment define a minimal effective dose of rhGAA, in combination with GalNAc-siRNA knockdown at ED80, for a long-term study as described below in Example 3.

### EXAMPLE 3

### Long-Term Efficacy of Combination Therapy with rhGAA and GalNAc-siRNA in GAA-KO Mice

To evaluate the long-term efficacy of combination therapy with rhGAA and GalNAc-siRNA in GAA-KO mice, a 12-week treatment is conducted in GAA-KO mice with weekly intravenous administration of rhGAA at the minimal effective dose (e.g., 5 mg/kg, as determined in Example 2) or at 20 mg/kg (standard dose) in combination with chronic GalNAc-siRNA knockdown at the maximal knockdown dose (ED80). The dosing interval for repeated GalNAc-siRNA administration is guided by the results determined in Example 1. Diphenhydramine (15 mg/kg) is intraperitoneally injected 10-15 min prior to each enzyme administration to prevent anaphylactic reactions (Joseph et al. (2008) Clin. Exp. Immunol. 152(1):138-146). There are 6 groups with n=10 mice per group (total 60 mice).

Group 1: Repeated administration of GalNAc-siRNA+weekly ERT at 20 mg/kg

Group 2: weekly ERT only at 20 mg/kg

Group 3: Repeated administration of GalNAc-siRNA+weekly ERT at 5 mg/kg

Group 4: weekly ERT only at 5 mg/kg

Group 5: Repeated administration of GalNAc-siRNA only

Group 6: No treatment controls

Treatment starts at 3 months of age. Blood is collected every month to (1) monitor anti-rhGAA antibodies by ELISA, and (2) assess safety of M6PR knockdown by testing activities of liver enzymes (ALT and AST) and concentrations of glucose (hypoglycemia) and IGF2. Functional improvement is evaluated by testing Rota-rod performance, Wire-hang, and treadmill at ages of 4.5 and 6 months. Urine is collected prior to euthanasia for testing urinary glucose tetrasaccharide Glc4, a biomarker of Pompe disease, by liquid chromatography-stable isotope dilution tandem mass spectrometry (LC-MS/MS) (Young et al. (2009) Med. Genet. 11(7):536-541). All mice are euthanized 24 hours after the last (12^{th}) rhGAA injection following overnight fasting to collect tissues. Correction of glycogen storage is assayed biochemically (glycogen content) and histologically (PAS-staining) in liver, heart, quadriceps, gastrocnemius, and diaphragm. M6PR expression is assessed in liver and muscle by Q-RT-PCR, Western blot, and IHC.

Potential impact of hepatic M6PR suppression on intracellular traffic of liver lysosomal enzymes is assessed by comparing levels of up to three putative lysosomal marker enzymes, acid phosphatase (AP), N-acetyl-beta-D-glucosaminidase (beta-NAG), and beta-galactosidase (beta-Gal), in livers of GalNAc-siRNA-treated mice (Group 5) to no-treatment controls (Group 6).

### EXAMPLE 4

### Long-Term Efficacy of rhGAA in Combination with Lower Doses of GalNAc-siRNA in GAA-KO Mice

If there is clear evidence of improved ERT efficacy with M6PR knockdown but side effects at the maximal M6PR suppression are observed in Example 3, lower doses (e.g., ED50 and ED30) of GalNAc-siRNA treatment are tested with the optimal ERT concentration as determined in Example 3. To evaluate the long-term efficacy of rhGAA in combination with lower doses of GalNAc-siRNA in GAA-KO mice, a 12-week study is conducted similar to the experiment in Example 3, including following 4 groups with n=10 mice per group (total 40 mice).

Group 1: Repeated administration of GalNAc-siRNA at ED50+weekly ERT at 20 mg/kg

Group 2: Repeated administration of GalNAc-siRNA at ED50 only

Group 3: Repeated administration of GalNAc-siRNA at ED30+weekly ERT at 20 mg/kg

Group 4: Repeated administration of GalNAc-siRNA at ED30 only

Sample collection and analysis, functional testing, and potential side effect of GalNAc-siRNA-mediated M6PR knockdown is assessed as described in Example 3. ERT only (20 mg/kg) and no treatment controls are already included in Example 3.

Any patents or publications mentioned in this specification are indicative of the levels of those skilled in the art to which the invention pertains. These patents and publications are herein incorporated by reference to the same extent as if each individual publication was specifically and individually indicated to be incorporated by reference.

One skilled in the art will readily appreciate that the present invention is well adapted to carry out the objects and obtain the ends and advantages mentioned, as well as those inherent therein. The present examples along with the methods described herein are presently representative of preferred embodiments, are exemplary, and are not intended as limitations on the scope of the invention. Changes therein and other uses will occur to those skilled in the art which are encompassed within the spirit of the invention as defined by the scope of the claims.

We claim:

1. A method of improving the efficacy of an enzyme replacement therapy for a lysosomal storage disease in a subject comprising administering to the subject a therapeutically effective amount of a therapeutic candidate such that the efficacy of the enzyme replacement therapy is enhanced.

2. The method according to claim 1, wherein the therapeutic candidate is RNAi polynucleotides or small molecule drugs.

3. The method according to claim 1, wherein the therapeutic candidate is selected from the group consisting of double stranded RNA, antisense oligonucleotides (ASO), small interfering RNA (siRNA), short hairpin RNA (shRNA), microRNA (miRNAs) oligonucleotides, and small molecule drugs.

4. The method according to claim 1, wherein the therapeutic candidate is delivered to liver cells with a viral vector, selected from the group consisting of an adenoviral vector, an adeno-associated viral (AAV) vector, a retrovirus vector, a hemagglutinating virus of Japan (HVJ) vector, a lentiviral vector, and a hepatitis B virus vector.

5. The method according to claim 1, wherein the therapeutic candidate is conjugated to one or more moieties used to target the therapeutic candidate to liver cells or is encapsulated in a delivery vehicle.

6. The method according to claim 5, wherein the delivery vehicle is conjugated to one or more moieties used to target the therapeutic candidate to liver cells.

7. The method according to claim 5, wherein the delivery vehicle is a PEGylated liposome or nanoparticle, oligonucleotide nanoparticle, cyclodextrin polymer (CDP)-based nanoparticle, biodegradable polymeric nanoparticle formulated with poly-(D,L-lactide-co-glycolide) (PLGA), Poly-lactic acid (PLA), or N-(2-hydroxypropyl)methacrylamide (HPMA), lipid nanoparticle (LNP), stable nucleic acid lipid particle (SNALP), or vitamin A coupled lipid nanoparticle.

8. The method according to claim 5, wherein the therapeutic candidate is chemically conjugated to one or more moieties that bind to an asialoglycoprotein receptor (ASGPR), an apolipoprotein, or a glycosaminoglycan.

9. The method according to claim 5, wherein the therapeutic candidate is chemically conjugated to one or more N-acetylgalactosamine (GalNAc) molecules.

10. The method according to claim 1, wherein the therapeutic candidate is capable of down-regulating the expression of mannose-6-phosphate receptor (M6PR) in a liver-specific manner in the subject.

11. The method according to claim 1, wherein the therapeutic candidate is an M6PR-specific short interfering RNA (siRNA) chemically conjugated to GalNAc.

12. The method according to claim 1, wherein the lysosomal storage disease is Fabry disease, Gaucher disease, Hurler disease, Hurler-Scheie disease, Scheie syndrome, Hunter disease, Maroteaux-Lamy syndrome, Pompe disease, Niemann Pick B, Batten, or Wolman disease.

13. The method according to claim 1, wherein the therapeutic candidate is administered intravenously, subcutaneously, transdermally, intradermally, intramuscularly or orally.

14. The method according to claim 1, wherein the therapeutic candidate is administered concurrently with, prior to, or subsequent to the enzyme replacement therapy in the subject.

15. The method according to claim 1, wherein the therapeutic candidate is administered concurrently with, prior to, or subsequent to the enzyme replacement therapy with rhGAA in the subject.

16. The method according to claim 1, wherein the therapeutic candidate and the enzyme replacement therapy are administered to the subject on an effective dose level and dosing interval basis.

17. The method according to claim 1, wherein the therapeutic candidate is administered concurrently with, prior to, or subsequent to the enzyme replacement therapy with rhGAA in the subject, and wherein the therapeutic candidate is an M6PR-specific siRNA chemically conjugated to GalNAc or an M6PR-specific siRNA encapsulated in a delivery vehicle.

18. A method of inhibiting expression of an M6PR mRNA comprising administering to a subject undergoing an enzyme replacement therapy for a lysosomal storage disorder an effective amount of a therapeutic candidate, wherein the therapeutic candidate comprises an M6PR-specific RNAi polynucleotide.

19. The method according to claim 18, wherein the therapeutic candidate is chemically conjugated to one or more N-acetylgalactosamine (GalNAc) molecules or encapsulated in a delivery vehicle.

20. The method according to claim 18, wherein the therapeutic candidate is an M6PR-specific short interfering RNA (siRNA) chemically conjugated to GalNAc or an M6PR-specific siRNA encapsulated in a delivery vehicle.
